(19) United States
(12) Patent Application Publication
(10) Pub. No.: US 2008/0004522 A1
(43) Pub. Date: Jan. 3, 2008

(54) Method in Mri-Imaging and Mri Apparatus with a Triggering Device

(76) Inventors: Taija Finni, Jyvaskyla (FI); Sulin Cheng, Jyvaskyla (FI); Jussi-Pekka Usenius, Muurame (FI); Marko Havu, Jyvaskyla (FI)

Correspondence Address: Fildes & Outland, 20916 Mack Ave Ste, Grosse Pointe Woods, MI, 48236, US

(21) Appl. No.: 11/667,520

(22) PCT Filed: Dec. 15, 2005
(86) PCT No.: PCT/FI05/50462

§ 371 Date: May 10, 2007

(30) Foreign Application Priority Data
Dec. 15, 2004 (FI) 20045484

(51) Int. Cl. A61B 5/055

(52) U.S. Cl. 600/415

(57) ABSTRACT

The invention relates to MR imaging method enabling velocity encoded cine phase contrast (VE-PC) image acquisition of muscle tissue velocities during dynamic muscle contraction. Method comprises step of:
a limb of a subject is inserted into the coil (13) of a MR scanner having a field of a view (FOV), the subject uses muscles in the field of view (FOV) causing a join movement of the limb,
a synchronizing means (20, 35) are provided to contract the muscles in a predetermined cycle and the phase of the movement is detected,
a set of MR images are provided during the cycle, where the MR image acquisition is triggered by the synchronizing means (20, 35).

### BACKGROUND OF THE INVENTION 1. Field of the Invention

The invention relates to MR imaging method and a MRI apparatus enabling velocity-encoded cine phase-contrast (VE-PC) image acquisition of muscle tissue velocities during dynamic muscle contraction, in which method
a limb of a subject is inserted into the coil (13) of a MR scanner having a field of a view (FOV)
the subject uses muscles in the field of view (FOV)
the subject is guided to contract the muscles in a cyclic way forming cycles of the contraction
a phase of each cycle is determined and phase data is obtained
a set of MR images, each image presenting a chosen phase, are provided during the cycle, where the MR image acquisition is synchronized with the phase data,

2. Description of the Prior Art

Typical MR images used for diagnostic purposes in radiology visualize and differentiate between different types of human (organic) tissue by virtue of the differences in density of protons within them, and their microscopic magnetic relaxation properties. The protons which are detected in MRI need to be able to diffuse relatively freely (such as in muscle) as opposed to those within solid structures (such as dense cortical bone or tendons). In solid structures, even though there are protons, the static nature of these protons tends to broaden the spectral characteristics of the signal emanated to an extent that these protons cannot be detected. As a consequence, these structures are depicted as signal voids.

Protons which are moving rapidly, either coherently or randomly, are not visualized in typical MR images, appearing more as image artifacts. However, special imaging techniques can be used in which the MR image can be made sensitive to coherent motion of the protons, such as exists in flowing blood, movement of the cardiac muscles (myocardium), or repeated isometric contractions of the muscle. Such specialized imaging techniques include velocity-encoded phase-contrast scans, in which the velocity of protons moving with a coherent velocity is quantified by means of the phase dispersion they generate in the signal detected. Velocity-encoded cine phase-contrast imaging has been used for measurement of dynamics of muscle contraction (Drace, J. E., Pelc, N.J. Measurement of skeletal muscle motion in vivo with phase-contrast MR imaging. JMRI 4:157-163, 1994, Finni et al. 2003, Sinha et al. 2004).

The scanners have means to synchronize the imaging with a phase of the cardiac or respiratory cycle. This is called gating, and it can be done either prospectively or retrospectively. Gating combines 1D scan lines to form a temporal series of one or more 2D images. If only a single scan line were to be acquired over one cycle, a series of 20 images with 128 lines each would take 2560 cycles to complete. Fortunately there are scanner sequences that enable acquiring multiple image lines during one cycle. Usually about 60 cycles are enough to complete a series of 20 images. Alternatively, real-time echo planar imaging can be used to construct the entire series during one cycle.

The validity of the phase-contrast method has been reported earlier (Drace & Pelc 1994, Lingamneni, A, Hardy, Pa., Powell, K A, Pelc, N.J. Validation of cine phase-contrast MR imaging for motion analysis. JMRI 5:331-338, 1995. Sinha et al 2004).

Velocity-encoded phase-contrast MRI provides two sets of images of, for example, 20 temporal phases during one cycle. One cycle is defined as time between consecutive QRS-complexes in cardiac imaging, or the time of one complete contraction-relaxation cycle during muscle work. Of the two sets of the images, one set contains velocity information (phase-contrast images) and the other anatomical information (magnitude images). In each velocity image the grayscale value of the voxel can be converted to represent the absolute velocity using VENC and VENC-scale of the tissue presented by the voxel (volumetric area determined by the field of view in the acquisition). The tissue movements during the cycle can be tracked using the velocity information in the 20 phase-contrast images.

Previously, the skeletal muscle movement in humans using VE-PC MRI has been done during isometric muscle contractions, i.e. in conditions where no joint movement occurs. For example, Finni et al. (2003a,b) and Sinha et al. (2004) have acquired data from lower leg using a posterior half of a fibreglass cast 6 that immobilized the ankle at an angle of 90°, see FIG. 9. An optical interferometer 7 (Fiberscan 2000, Luna Innovations, Va.) was embedded in the sole of the cast for measurement of plantarflexion force exerted by the foot on the cast. Both legs were inserted into the head coil 13 of a MRI scanner 10. Lying in supine position inside the scanner tube 11 the subject was asked to perform ˜70 isometric contractions using the plantarflexor muscles. The subject was requested to synchronize the isometric contraction-relaxation cycles to a computer generated audio cue that was fed through the scanner audio system. Instantaneous information of the produced force level was displayed on a LED bar enabling the subject to match the targeted force level.

The strain exerted on the cast was measured as changes in the length of the optical cavity that formed a part of a Fabry-Perot interferometer. The strain signal was also used to activate a set of LED bars placed at the opening of the magnet bore, enabling the subjects to monitor the level of force being exerted. Additionally, the initial rise in the signal was used to trig the MRI acquisition. The output of the force transducer was electronically shaped to produce a simulated ECG pulse at a particular threshold, which was fed to the cardiac gating system, triggering each segment of phase-encoding level to the beginning of the force rise.

U.S. Pat. No. 5,772,595; Votruba et al. 1998, Multipositional MRI for kinematic studies of movable joints, describes a method for taking images of still joints in different positions, and sequencing the movement of the joint to a set of predetermined positions. A series of images is produced during repeated muscle contractions, but each image presents the muscle in a static position. Votruba does not actually detect the position of the movable joint when the automatic device is set to place the joint in a set of predetermined positions.

The article Asakawa et al. 2003, “Cine Phase-Contrast Magnetic Resonance Imaging as a Tool for Quantification of Skeletal Muscle Motion” relates to imaging muscles in motion. This article is a review on the use of cine phase-contrast magnetic resonance imaging for quantification of skeletal muscle motion. It describes a set-up for imaging the motion of the thigh muscles during knee flexion and extension. They use the beginning of the movement to trig the scanning, whereas our design allows for synchronisation of imaging and any muscle-contraction-related magnitude (e.g. displacement, force, muscle activation, rate of blood flow) throughout the contraction cycle. Another article of Asakawa et al. 3. 2003, “Real-Time Imaging of Skeletal Muscle Velocity” presents a similar method. In that study they tested the feasibility of using real-time phase-contrast MRI to track velocities of skeletal muscle motion. It describes a set-up for imaging the motion of the biceps brachii and triceps brachii muscles during elbow flexion and extension. They use the beginning of the movement to trig the scanning, whereas our design allows for synchronisation of imaging and any muscle-contraction-related magnitude (e.g. displacement, force, muscle activation, rate of blood flow) throughout the contraction cycle.

### SUMMARY OF THE INVENTION

The invention provides a MR imaging method enabling velocity-encoded cine phase-contrast (VE-PC) image acquisition of muscle tissue velocities during dynamic muscle contraction, i.e. in conditions where joint movement occurs. The method applies to imaging of skeletal, bone and tendinous tissues. The invention provides also a MRI apparatus with triggering means.

Though any limb having a joint may be imaged, usually a leg is used.

Where electronic cables are not suitable inside MRI-tube, fibre optics is used instead.

According to the invention the phase of a cycle is detected continuously or at least essentially continuously i.e. in many points in a cycle. The phase data is added to MRI image data or used for determining which phase the current scan line is to be associated with. It has been found that continuous detection of phase is necessary when comparing that to triggering from one point of a cycle. Many subjects could not maintain a constant pace in cyclic movements. When using a single point of cycle to trig the scanning, the quality of the images decreased dramatically towards the end of the cycle.

When multiple scan-line MR imaging is used, and that the number of the cycles is 2-4 times the number of phases in a cycle.

In the following, the invention is examined with reference to the accompanying drawings, which show some embodiments of the invention.

### BRIEF DESCRIPTION OF THE DRAWINGS

FIG. 1 shows the MRI hardware as whole.

FIG. 2 shows a setup with a light detector for measuring thigh muscles with knee extension-flexion movement.

FIG. 3 shows a setup with a light detector for measuring back muscles from lumbar region with hip extension-flexion movement.

FIG. 4 shows a setup with a pneumatic detector for measuring calf muscles with ankle plantarflexion-dorsiflexion movement.

FIG. 5 shows the general description of the setup in the MR environment according to one embodiment of the invention using a pneumatic detector.

FIG. 6 shows the details of the device for support and resistance as well as the light detector.

FIG. 7 shows the trigger detection unit for a light detector.

FIG. 8 shows the optical metronome control unit.

FIG. 9 shows a measuring arrangement according to the prior art.

### DETAILED DESCRIPTION OF THE PREFERRED EMBODIMENTS

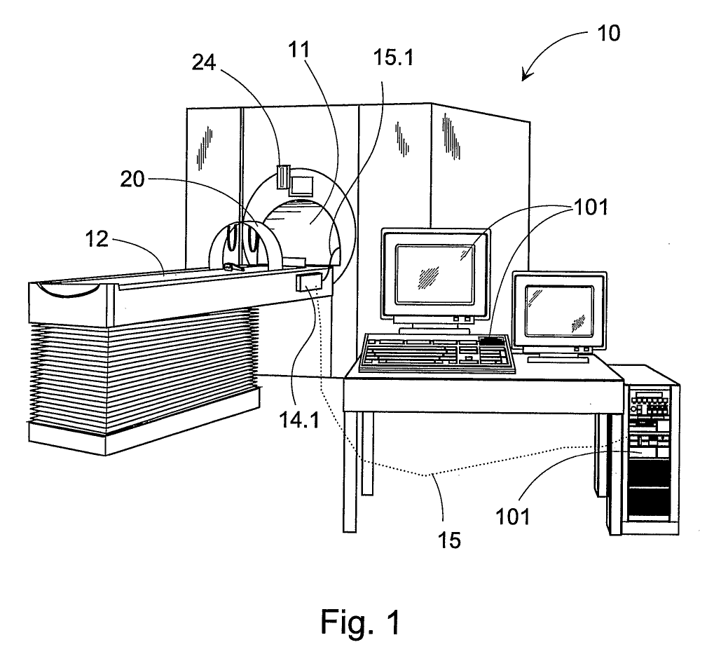

In FIG. 1 the MRI device is referred to with reference number 10 and it comprises of a big magnet (not shown) encompassing MRI bore 11, gradient and RF-coils (not shown), table 12 for a subject (patient). The main computer 101 with user interface devices (displays, keyboard) is also part of the system and it is located in another room called an operating room while MRI bore 11 and table 12 are in a scanning room. Trigger and detection unit 14.1 is connected with an internal cable to main computer 101 of the MRI device. In this embodiment a pneumatic sensor is used for the detection of phase. The pneumatic sensor of unit 14.1 itself is a standard fitting but is used in a new way as described later.

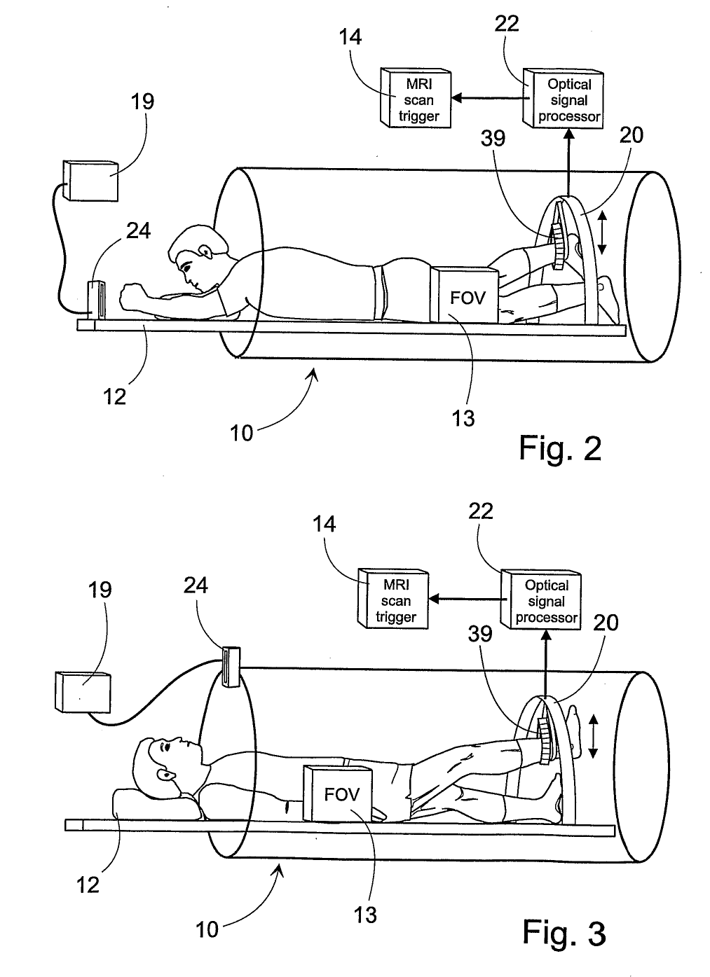

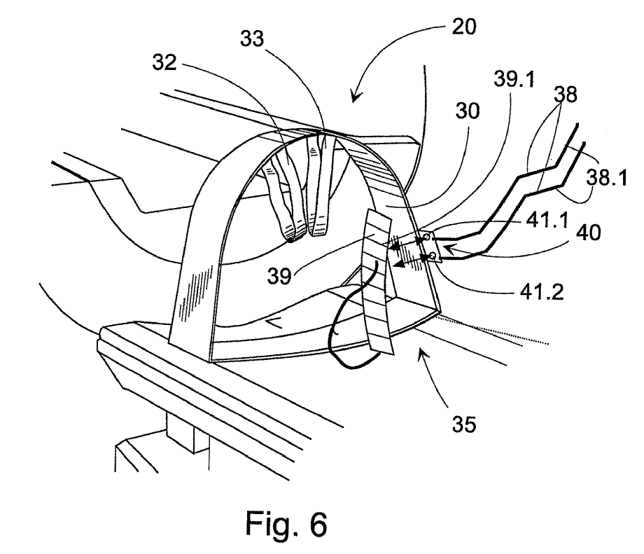

FIG. 2 shows a setup for measuring thigh muscles with knee extension-flexion movement. The subject is lying in prone position on the table 12. The optical metronome 24 is placed in front of the subject to enable him to see the light signal. His ankle is attached to the resistive device 20 and to the target plate in the detecting device 39 (shown in detail in FIG. 6). The coil 13 is placed in the region (thigh) where the images are acquired.

FIG. 3 shows a setup for measuring back muscles from lumbar region with hip extension-flexion movement. The subject is lying in supine position on the table 12. The optical metronome 24 is placed in front of the subject to enable him to see the light signal. His ankle is attached to the resistive device 20 and to the target plate in the trigger device. The coil 13 is placed in the region (lower back) where the images are acquired.

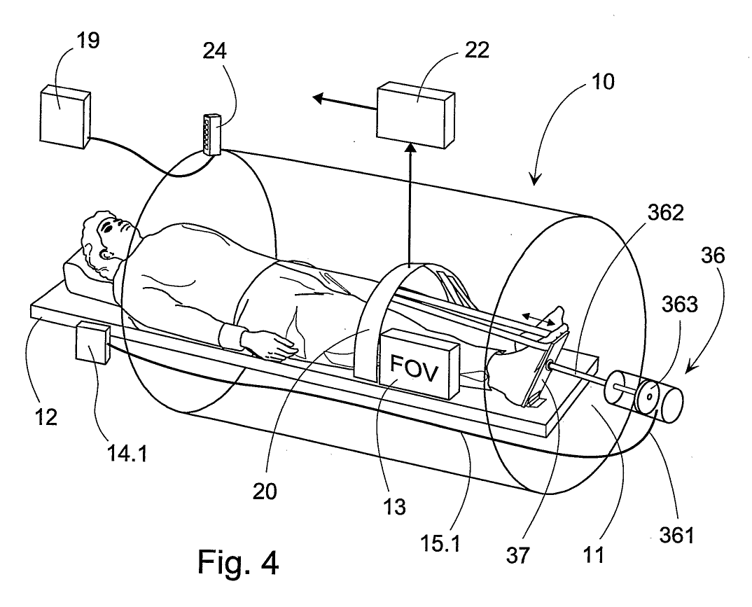

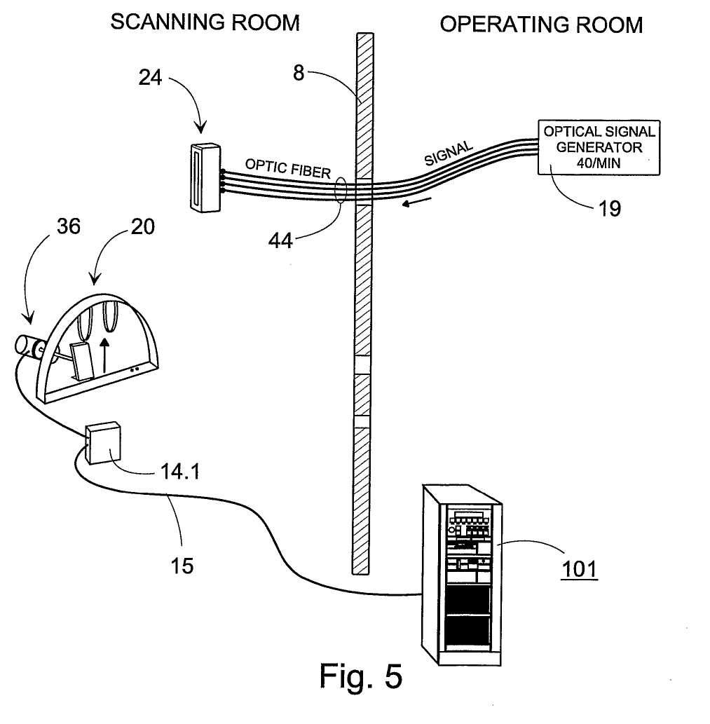

FIG. 4 shows a setup for measuring calf muscles with ankle plantarflexion-dorsiflexion movement. A schematic pneumatic sensor for detecting phase is shown. The subject is lying in supine position on the table 12. The optical metronome 24 is placed in front of the subject to enable him to see the light signal. His ankle is attached to the resistive device 20 and to the lever 35 of the pneumatic member in the detection device. The sole of the subject exerts the pedal 35, which is articulated onto the table 12. The pedal 37 moves the rod 362 of the piston 363 in the cylinder 36. This gives a pressure signal to trigger and detection unit 14.1, which gives an electrical signal to the main MR computer, signal being relative to the actual phase of the cycle. The coil 13 is placed in the region (thigh) where the images are acquired.

FIG. 5 shows a general description of the setup in the MR environment between MRI room and an operating room, when a pneumatic sensor is used. The optical metronome control unit 19 is placed in the operating room next to the MRI room and it is connected with an optic fibre series 44 (bundle) through a wall 8 to the optical metronome 24 that comprises of a bundle of LED's in one column. The visual cue for rhythm also indicates the phases of movement with rising and descending light bar consisting of individual fibre ends.

The pneumatic cylinder 36 with its piston creates pressure, which is detected by unit 14.1. This sends the phase data as an electrical signal to main computer 101. The phase data is used for determining which phase the current scan line is to be associated with, or stored with the image data. The trigger output is fed to the scanner via cardiac gating system using the scanner's own interface.

There are two main processing ways to build up MR images of the cyclic movements with 1D scanning. According to the first process the phase data is used for triggering the scanning and the lines acquired at a certain point of the cycle are combined directly to build up the whole picture. The advantage is minimum number of required scanned line images.

In the second process where the triggering follows a predetermined cycle after timing pulse (one per cycle) and phase data is stored with the image data, each line image having actual phase diverging nominal phase (too much) is rejected and next scanned image with same phase is taken instead. The latter means that there should be enough extra cycles of data to compensate rejected images.

If selective excitation techniques are employed, adjacent lines or planes can be imaged while waiting for the relaxation of the first line or plane toward equilibrium. This decreases the total image acquisition time. With the use of standard multiple slice imaging and a spin echo pulse sequence, a number of slices at several anatomical levels can be acquired over one cycle. When using a gradient echo pulse sequence, either multiple phases of a single anatomical level or several slices at different anatomical levels can be acquired over one cycle.

FIG. 6 shows the details of the device 20 for support and resistance as well as a light sensor. The elastic bands 32, 33 attached to the frame 30 provide the resistance for the movements. The resistance bands 32, 33 have known and different stiffness. The light sensor 35 is herewith introduced. A laser transceiver element 40 has been attached to the frame 30 of the support device 20. The frame 30 has several attachment sites allowing flexible configurations for identification of movement phases. A target plate 39 with lines 39.1 is to be fixed to a leg of the subject. The lines are spaced with a different space as compared to that of the laser beams. This gives easy way to indicate the direction of the movement. The lines give pulses to the reflection of each laser beam transmitted from laser transmitter. Each reflection is read by a light sensor. Transmitters and sensors are at a distance in a detection unit, when they are connected to the element 40 by light fibers 38 and 38.1.

The continuous detection of the cycle phase may take place in several ways. One embodiment uses a bundle of fibres, each fibre detecting a certain angle. Another embodiment has only one pair of fibres as above, but the light is split for many holes in different angles, where holes have different sizes in the lever and the frame. The angle is detected thus according to the magnitude of light signal.

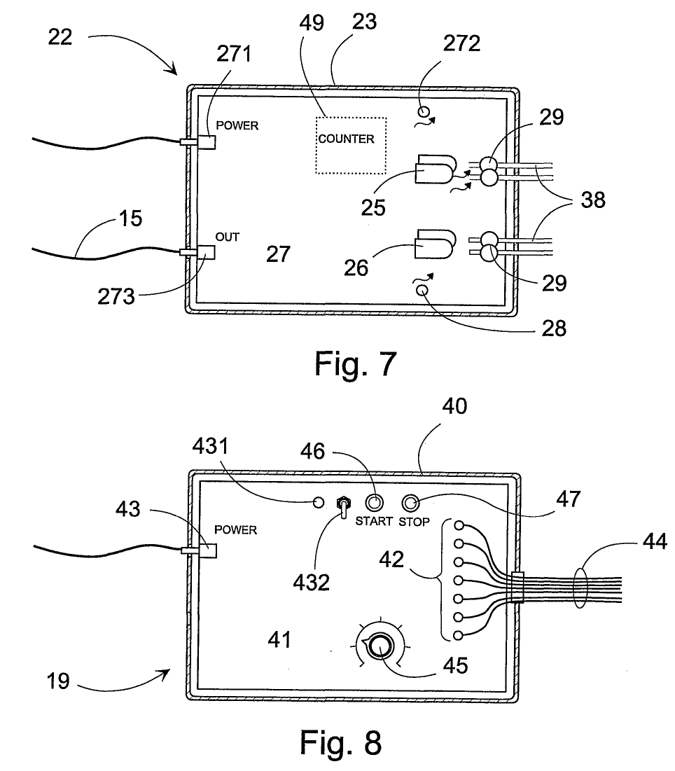

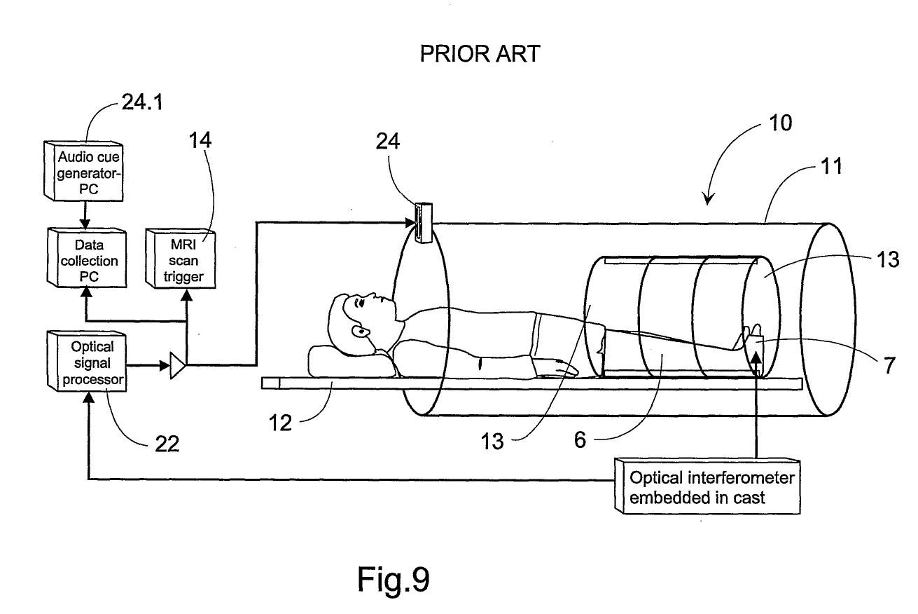

FIG. 7 shows the detection unit 22 schematically. It is assembled into circuit board 27 in a box 23. Only the relevant components are shown on the circuit board here. The laser transceiver element 40 (FIG. 6) is connected with the optic fibres 38, 38.1 to the unit 22. The ends of the fibres 38, 38.1 are secured by the locks 29 against the transmitters 25, 25.1 (laser/LED) and receivers 26, 26.1, respectively. A special counter 49 counts pulses from the light sensors. The actual position is determined continuously.

The power source is connected to the power input 271 and a power LED 272 shows an indication about a power supply. An indication LED 28 blinks whenever detection is made. A phase detection signal according to the pulse counter 48 is output from the connector 273 to the wire 15.

FIG. 8 shows the optical metronome control unit 19. The metronome creates rising and descending light signal with a series 42 of 12 LED's that light up as a vertical bar. The frequency of the signal can be varied by the potentiometer 45. The rest-work cycle can be flexibly determined. The metronome also creates a trigger output signal consisting of a square wave pulse with the same frequency as the light signal (connector 49). The power source is connected to the power input 43. A power switch 432 and a power LED 431 are provided on the board. Pushing the start button 46 causes the optical metronome to start and the stop button 47 stops it.

It is to be understood that the above explanation and the related figures are only meant to illustrate a MRI apparatus and a trigger device of the present invention on view. The invention has not thus been limited solely to the embodiments presented above or defined in the claims, but the several different variations and versions that are possible within the frames of the inventive idea defined by the accompanying claims will be obvious to any specialist in the field.

1. MR imaging method enabling velocity-encoded cine phase-contrast (VE-PC) image acquisition of muscle tissue velocities during dynamic muscle contraction, in which method
a limb of a subject is inserted into the coil of a MR scanner having a field of a view (FOV);
the subject uses muscles in the field of view (FOV);
the subject is guided to contract the muscles in a cyclic way forming cycles of the contraction;
the muscles are contracted relating to a joint movement of the limb and a dynamic muscle contraction is achieved;
a phase of each cycle is determined and phase data is obtained;
a set of MR images, each image presenting a chosen phase, are provided during the cycle and stored, where the MR image acquisition is synchronized with the phase data,
characterized in that the phase of the cycle is detected essentially continuously and image data having same phase is combined.

2. Method according to claim 1, characterized in that multiple scan-line MR imaging is used, and that the number of the cycles is 2-4 times the number of phases in a cycle.

3. Method according to claim 1, characterized in that the actual phase of the cycle is detected by a pressure sensor of a pneumatic device exerted by the limb movement.

4. Method according to claim 1, characterized in that the actual phase is detected by a light sensor that directly senses the position of the limb.

5. MRI apparatus enabling velocity-encoded cine phase-contrast (VE-PC) image acquisition of skeletal muscle tissue velocities during dynamic muscle contraction, in which MRI apparatus includes
MRI scanner with a coil adapted to receive a limb of a subject
said coil having a field of view for muscles of the limb
means for guiding subject for a cyclic contraction of the muscles
means for determining the phase of a cycle of a muscle contraction
MR image acquisition system to obtain and store a set of MR images presenting a whole cycle as predetermined phases
means for synchronizing the acquisition system to the cycle of the muscle contraction, characterized in that
the means for determining the phase of the cycle includes a detector for a limb movement, which detector is adapted to detect the phase of the movement essentially continuously, and the MRI apparatus is adapted to combine image data having same phase.

6. MRI apparatus according to claim 5, characterized in that the detector for limb movement is adapted directly to trigger the acquisition system.

7. MRI apparatus according to claim 5, where line-based scanning is used, characterized in that the MR image acquisition system is adapted to combine stored images of same phase and reject images in favour of next stored image when the stored phase differs from the predetermined phase.

8. MRI apparatus according to claim 5, characterized in that the detector is a light sensor.

9. MRI apparatus according to claim 5, characterized in that the detector is a pressure sensor and a pneumatic device exercised by the limb.

10. MRI apparatus according to claim 6, characterized in that the detector is a light sensor.

11. MRI apparatus according to claim 6, characterized in that the detector is a pressure sensor and a pneumatic device exercised by the limb.

12. Method according to claim 2, characterized in that the actual phase of the cycle is detected by a pressure sensor of a pneumatic device exerted by the limb movement.

13. Method according to claim 2, characterized in that the actual phase is detected by a light sensor that directly senses the position of the limb.
